(19) United States
(12) Patent Application Publication
(10) Pub. No.: US 2008/0021335 A1
(43) Pub. Date: Jan. 24, 2008

(54) Intracerebral Blood Flow Measuring Device

(76) Inventor: Hideki Harada, Fukuoka (JP)

Correspondence Address: WENDEROTH, LIND & PONACK, L.L.P., 2033 K STREET N. W., SUITE 800, WASHINGTON, DC, 20006-1021, US

(21) Appl. No.: 10/582,612

(22) PCT Filed: Dec. 9, 2004
(86) PCT No.: PCT/JP04/18364

§ 371 Date: Apr. 16, 2007

(30) Foreign Application Priority Data
Dec. 12, 2003 (JP) 2003-414819

(51) Int. Cl. A61B 5/026

(52) U.S. Cl. 600/504

(57) ABSTRACT

It is intended to standardize MCAO model and to further improve the reproducibility and reliability thereof. There is provided a probe holding device (10) which includes a probe holding member (14) for holding a blood flowmeter probe (12), and such device is used together with the blood flowmeter probe when measuring intracerebral blood flow. While holding the blood flowmeter, the probe holding member can be disposed in a position of being adjacent to and outside temporal bones.

### TECHNICAL FIELD

The present invention relates to a device which measures a blood flow such as a blood flow rate in a brain, and particularly in a brain portion which is located closely to a temporal bone(s). The present device is preferably applicable to intracerebral blood flow measurement in various experiments in which animals such as rats, mice or the like are used.

### BACKGROUND ART

Middle Cerebral Artery Occlusion, MCAO) model was proposed by Koizumi and Zea Longa et. al. in which a rat was used with an intraluminal filament, and such model has been used for the mechanism analysis as well as new therapy research and development as to ischemic cell death in brain (see Non-Patent References 1 and 2 mentioned below). This model is advantageous in that it is relatively easily carried out and less-invasive, and that craniotomy which affects blood-brain barrier permeability, brain pressure, and brain tissues is not required.

However, a trauma size of a rat used for the experiment, a subarachnoid hemorrhage rate by means of blood vessel perforation and an early mortality rate between 24 hours and 48 hours after the model preparation depend on a person who carries out the experiment, namely experiment reproducibility of the model is poor. This means that statistic consideration is required in which many animals are used with a lot of costs as well as a lot of time.

It is contemplated that the poor reproducibility would be caused by ischemic interval difference, reperfusion timing difference, filament quality difference, filament allocation difference and so on. Then, it is necessary that ischemia is confirmed by the measurement of a regional cerebral blood flow (rCBF). It has been proposed that using the Laser-Doppler Flowmetry (LDF) is effective for such measurement to use, so that the reliability of the model is improved (see Non-Patent Reference 3 mentioned below). However, when the LDF is used, a special treatment is required.

Specifically, a blood flow rate is measured by contacting a laser radiating portion of a LDF probe with an exposed dura mater after craniotomy is performed on an anesthetized rat so as to remove a top portion of cranial bones. In order to fix the probe to a cephalic portion of the rat, the probe is secured to the cranial bones using a biocement.

In order to fix the probe to the cephalic portion of the rat after the craniotomy as described above, extremely precise technique and time are essential. Particularly, when the number of rats to be examined is large, a time constraint matter also occurs.

### Non-Patent Reference 1:

Koizumi J, Yoshida Y, Nakazawa T, Ooneda G. Experimental studies of ischemic brain edema, I: a new experimental model of cerebral embolism in rats in which recirculation can be introduced in the ischemic area. Jpn J Stroke. 1986; 8:1-8

### Non-Patent Reference 2:

Longa E Z, Weinstein P R, Carlson S, Cummins R.

Reversible middle cerebral artery occlusion without craniectomy in rats. Stroke. 1989; 20:84-91

### Non-Patent Reference 3:

Schmid-Elsaesser R, Zausinger S, Hungerhuber E, Baethmann A, Reulen H J. A critical reevaluation of the intraluminal thread model of focal cerebral ischemia:

evidence of inadvertent premature reperfusion and subarachnoid hemorrhage in rats by laser-doppler flowmetory. Stroke. 1998; 29:2162-70

### DISCLOSURE OF THE INVENTION

### Problem to be Solved by the Invention

Thus, it is an object of the present invention to standardize the MCAO model and improve its reproducibility and reliability further.

### Means to Solve the Problem

As a result of intensive studies about the above problem, it has been found that blood flow measurement can be carried out with improved reproducibility and reliability and without damaging cranial bones as in the prior art when a probe for a blood flowmeter which uses the Doppler effect (which is hereinafter referred to as a “blood flowmeter probe”) is provided between a temporal muscle and a temporal bone. Based on this finding, the inventor has completed the present invention which will be explained below.

It is noted that the blood flowmeter probe has been already known in the field of the blood flow measurement which relates to the medical care, medical science and so on, and it is intended to mean that the blood flowmeter probe is a probe which is used for a blood flowmeter well known as an apparatus which measures, as a blood flow, at least one of a blood flow rate, an amount of blood flow and a blood flow velocity with using the optical or sonic Doppler effect, particularly ultrasonic Doppler effect, and also which probe has a function to irradiate light or sound to an object of which blood flow is to be measured and also to receive light or sound reflected from the object. The blood flowmeter probe comprises a conductor(s) which transfers an electric signal and/or an electric power for the blood flow measurement.

In the first aspect, the present invention provides a probe holding device which includes a probe holding member for holding a blood flowmeter probe and which is used with the blood flowmeter probe when intracerebral blood flow is measured, and the probe holding member is characterized in that it is allowed to be disposed in a position of being adjacent to and outside a temporal bone(s) while the blood flowmeter probe is held by the member.

With the probe holding device according to the present invention, the probe holding member can be disposed in a position of being adjacent to and outside a temporal bone on at least one side of the cranial bones which cover a brain while holding the blood flowmeter probe. The blood flowmeter probe to be held irradiates light or sound (particularly, ultrasound) to the brain through the temporal bone and also receives light or sound (particularly, ultrasound) which is reflected by the brain. The probe holding member is preferably in the form of a sheet (i.e. a form of which dimension along its thickness direction is considerably smaller than the other dimensions), and also it preferably has a size (particularly a thickness) which allows the blood flowmeter probe to be located in a space defined by the temporal bone and the temporal muscle next to the temporal bone. The shape of the sheet may be any appropriate one, and for example, it may be a rectangular, square, oval, circle or the like. The shape of the sheet is desirably adapted to be received by such space. In the case wherein the probe holding member is in the form of the sheet, the blood flowmeter probe is preferably also of a thin form. When the cranial bones are exposed by incision into skin of head, and a temporal muscle which is in a position of being adjacent to a temporal bone is separated away from the temporal bone, the above mentioned space is spontaneously formed. Such space may be referred to as a “natural pocket.”

It is noted that the probe holding member may be in a form other than the sheet form as far as it can be received by the “natural pocket” and also it allows the blood flow measurement. For example, an animal (for example, a rat) having a standard weight (or a standard figure) is selected as a model used in experiments for the blood flow measurement; a silicone resin is poured into the “natural pocket” of the model and then the resin is cured followed by taking out the cured resin mass as a master model; and using the master model, a plastic material is molded to obtain a probe holding member. In this example, there is provided a probe holding member which just falls in (that is, just fits) the “natural pocket”, so that measurement with a further improved accuracy becomes possible. In fact, as to the animals which are used for the blood flow measurement, their standard weight are predetermined, and therefore, once the master model has been obtained and the probe holding members have been produced using such master model, such probe holding members can be commonly used in other experiments in which the same kinds of animals are used.

Thus, the probe holding member according to the present invention may be produced by a process which is characterized in that a master model which corresponds to a space which is defined by and between a temporal bone and a temporal muscle is obtained, and then a plastic material is molded based on the master model.

The master model can be obtained by pouring a curable material into the space defined between and by the temporal bone and the temporal muscle followed by curing the curable material in the space. Any appropriate material may be used as the curable material. For example, an inorganic material such as plaster, or an organic material such as a curable resin may be used. A preferable material is a photo-curable (or UV-curable) resin, and particularly a silicone resin. In this case, the master model can be obtained by pouring the curable resin into the “natural pocket”, followed by irradiating the resin with light.

In a preferable embodiment, the probe holding device according to the present invention is designed such that the probe holding members are located in a position being adjacent to the temporal bones on the both sides, wherein light or sound is irradiated to the brain from the blood flowmeter probe through the temporal bones on the both sides so as to measure the blood flow in the brain. In such embodiment, the probe holding device comprises two of the probe holding members as described above, so that each probe holding member can be placed adjacently to each temporal bone. The two probe holding members may be independent (that is, separated) from each other. Alternatively, the probe holding device may comprise a bridging part which bridges (or connects) the two probe holding members together so as to form a single device.

In one embodiment of the preferable device as above mentioned, the bridging part is also in the form of a sheet, and each of the probe holding members are connected together to an edge portion on each side of the bridging part. Particularly, the probe holding device preferably has a U-shaped cross section as a whole, wherein the bridging part corresponds to the bottom bar of the “U” shape and each probe holding member extends upward from each end of the bottom bar. A width (or a length) of the edge portion of the bridging part may be equal to or different from that of the probe holding member. The bottom bar of the U shape may be linear or not, and for example it may be curved.

The probe holding device of which cross section is U-shaped may be formed by folding a rectangular sheet (preferably a strip sheet) having a predetermined size to be the U-shape. In this case, the width of the edge portion of the bridging part can be equal to that of the probe holding member. As a sheet which is preferable for such folding, a plastic sheet (such as a polypropylene sheet, a soft celluloid sheet, a silicone resin or the like) and a metal sheet (such as a stainless steel sheet or the like) may be exemplified. As far as the effects of the present invention are provided, any sheet which is made of any appropriate material may be used. In place of folding the sheet material, the probe holding members may be bonded to the bridging part using any appropriate means such as screwing, an adhesive, welding or the like.

The probe holding member holds the blood flowmeter probe. The manner with which the probe holding member holds the probe is not particularly limited as far as the probe is kept in a condition to be located adjacently to the temporal bone. The adjacently located condition of the prove may be a condition in which the probe (particularly, a part thereof which irradiates light or sound and receives light or sound) is in directly contact with the temporal bone or opposed to the temporal bone across a small gap. The former condition is preferable. Specifically, in one embodiment, a concave portion which corresponds to (or which is in complementary to) the form of the probe is provided to a sheet which forms the probe holding member, and when the probe is placed in the concave portion, surfaces of the probe and the probe holding member are substantially flush with each other, so that the probe does not protrude above the probe holding member. In other embodiment, the surface of the probe (particularly, a part thereof which irradiates light or sound and receives light or sound) may be protrude above the surface of the probe holding member. Further, the probe holding member may be similarly provided with a concave portion which corresponds to a conductor connected to the probe.

In one preferable embodiment, the probe holding device according to the present invention holds also a temperature sensor. It is preferable that the temperature sensor can measure a temperature of the temporal bone, preferably a portion of a brain of which blood flow is to be measured, which is convenient since a temperature can be measured simultaneously upon the blood flow measurement. Specifically, it is preferable for the probe holding device that it can hold a temperature sensor having a rod form or a flat form as a whole. As to the holding of the temperature sensor, the holding of the bpf as described above may be applicable. It is noted that a temperature sensing part of the temperature sensor is generally minimal and for example it is dot like.

In one embodiment, the probe holding device according to the present invention is adapted to be used for animals such rats, mice or the like which are used for various medical researches. That is, the size of the probe holding member is such that it can be inserted in a space between a temporal bone and a temporal muscle of such animals. In a particularly preferable embodiment, the probe holding member is obtained as described above by obtaining a master model for the “natural pocket” and molding a plastic material based on the master model. The probe holding device according to the present invention is applicable to other animals (for example, dogs, rabbits, monkeys or other laboratory animals), in which the probe holding member(s) has a size such that the member(s) can be inserted into the space between the temporal bone and the temporal muscle of such animal and the bridging part if any may be a size as desired.

Further, in the embodiment wherein the holding device uses the two probe holding members, it is preferable that the bridging part connects the probe holding members together such that each of the two probe holding members are easily located adjacently to each of the both temporal bones. In this embodiment, it is preferable that a force acts such that the probe holding members get closer when the two probe holding members are located on the sides of the both temporal bones respectively. Specifically, it is preferable that the two probe holding members sandwich cranial bones while pressing them inward. For example, in the case of the probe holding device having the U-shaped cross section, a width of the U-shape, that is, a separation between the probe holding members is adapted to be equal or similar to, slightly larger than or slightly smaller than an average width of cranial bones of an intended animal. When slightly smaller, it is preferable that the probe holding members can be located beside the temporal bones while the device is being expanded a little against a resiliency of the material which constitutes the device.

In the second aspect, the present invention provides a blood flow measuring device (or blood flowmetry device) which comprises the probe holding device according to the present invention as described above. The blood flow measuring device is a device wherein the blood flowmeter probe(s) is in a position of being provided to the probe holding device as described above, and a conductor(s) runs out of the blood flowmeter probe(s). The conductor is required so as to irradiate light or sound, and also to receive light or sound followed by processing it as signals, and such conductor is well known. Thus, no particular explanation as to the conductor is necessary. It is preferable for the blood flow measuring device according to the present invention that the probe holding member further comprises the temperature sensor as described above. It is noted that the blood flowmeter probe and the conductor are well known in the field, for example in the LDF field, and conventional ones may be used.

In the third aspect, the present invention provides a method for measuring blood flow in a brain of an animal with using the blood flow measuring device as described above. Such method comprises disposing the probe holding member between the temporal muscle and the temporal bone of the animal using the blood flow measuring device according to the present invention, irradiating light or sound from the blood flowmeter probe to the brain, and receiving light or sound which has been reflected by the brain by means of the blood flowmeter probe. In this method, the blood flow can be measured similarly to the case in which the blood flow measurement is generally carried out with using a blood flowmeter except that the probe holding member is disposed between the temporal muscle and the temporal bone while using the blood flow measuring device according to the present invention.

In the method according to the present invention as described above, before disposing the probe holding member between the temporal muscle and the temporal bone, a natural pocket is formed by incising scalp above a temporal muscle and a temporal bone and exposing them so as to form the natural pocket. If necessary, after disposing the probe, the temporal muscle is moved toward the temporal bone while the probe is sandwiched by the temporal muscle and the temporal bone, and then the scalp may be sutured with a conductor of the probe extending outwardly.

After disposing the blood flow measuring device as described above, various experiments and/or treatments are performed with respect to the animal. For example, an animal is treated to form the MCAO model, the MCAO experiment can be carried out while measuring brain blood flow noninvasively.

### EFFECTS OF THE INVENTION

When the probe holding device according to the present invention is used, the blood flowmeter probe can be disposed between the temporal muscle and the temporal bone. Such disposition of the probe allows the brain blood flow to be measured with the reproducibility and the reliability. When such blood flow measurement is to be carried out, the preparation for the measurement is very simple compared with a conventional method in which a top portion of the cranial bones is removed and the dura mater is exposed. As a result, the Doppler blood flowmeter can be readily applied to the brain blood flow measurement.

Thus, since the brain blood flow conditions can be monitored online with using the Doppler blood flowmeter, even a less experienced examiner can carry out experiments with the MCAO model in which more sufficient occlusion is achieved with the improved reproducibility and reliability. As a result, occurrence of the subarachnoid hemorrhage due to an inappropriate or unaccustomed treatment during the experiments is drastically suppressed, so that significant experiments can be carried out with a small number of animals at a low cost in a short time.

### BRIEF DESCRIPTION OF THE DRAWINGS

FIG. 1 schematically shows a perspective view of a probe holding device according to the present invention.

FIG. 2 schematically shows the probe holding device according to the present invention when viewing the device along a direction of the arrow A shown in FIG. 1.

FIG. 3 schematically shows the probe holding device according to the present invention when seeing the device along a direction of the arrow B shown in FIG. 1.

FIG. 4 schematically shows the probe holding device of FIG. 1 when viewing the to of the device from its above.

FIG. 5 shows a representative recording of the rCBF measured by the LDF in the Example.

FIG. 6 shows dynamic changes of the rCBF measured by the LDF as to the second group in the Example.

FIG. 7 shows calculation results of lesion volumes of the cortex and the subcortex in Example.

FIG. 8 schematically shows a perspective view of a probe holding device according to the present invention, which is similar to the device shown in FIG. 1 and which further comprises a heating element in a bridging part.

### EXPLANATIONS OF REFERENCES

10 . . . probe holding device

12, 12′ . . . blood flowmeter probe

14, 14′ . . . probe holding member

16 . . . bridging part

18, 18′, 20, 20′ . . . edge portion

24, 24′ . . . conductor

26, 26′ . . . temperature sensor

28, 28′ . . . conductor

30 . . . sound or light irradiating and receiving portion

32, 32′, 34, 34′ . . . opening

40 . . . heating element provision area (shaded portion)

44 . . . temperature sensor

### MODES FOR CARRYING OUT THE INVENTION

The device according to the present invention will be explained with reference to an example wherein an LDF is used. When kinds of the probe and the blood flow meter are changed, such device can be similarly applicable to the ultrasonic-Doppler flowmetry.

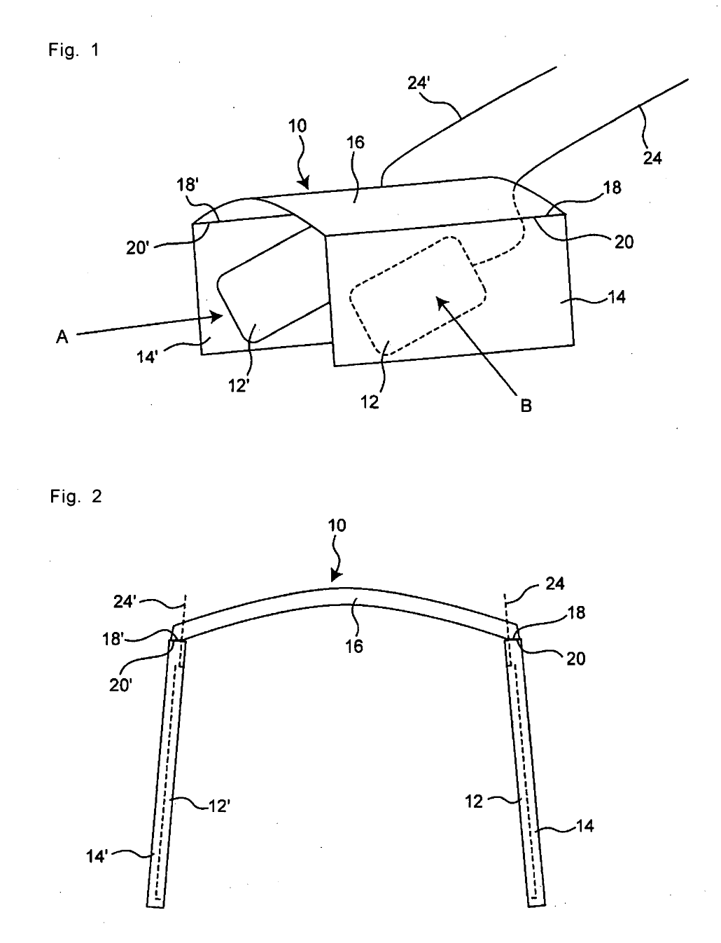

A probe holding device 10 according to the present invention is schematically shown in FIG. 1 in a perspective view. In the shown embodiment, blood flowmeter probes 12 and 12′ are located on the holding device 10, that is, the blood flow measuring device according to the present invention is shown.

The shown probe holding device 10 comprises two probe holding members 14 and 14′ which are connected together by means of a bridging part 16. As seen from the drawing, edge portions 18 and 18′ each of which corresponds to the width of the bridging part 16 are connected respectively along edge portions 20 and 20′ (in particular, along the whole lengths of the edge portions) together each of which corresponds to the width of each holding member. When viewing the device from the left side in FIG. 1 (see the arrow A in FIG. 1), the cross section of the probe holding members forms an inverse “U” shape, which corresponds to the configuration of the probe holding device 10. A distance between the probe holding members 14 and 14′ substantially corresponds to a distance between the temporal bones on the both sides of the skull. That is, the distance between the probe holding members is equal to the distance between the temporal bones, or slightly smaller than the distance between the temporal bones, but it can be opened a little due to a property of material(s) of the holding device, or slightly larger than the distance between the temporal bones but can be reduced a little due to a property of the material(s) of the holding device.

It is noted that a thickness of the probe holding members 10 is omitted in the embodiment shown in FIG. 1. As shown, the probe holding members 14 and 14′ include blood flowmeter probes 12 and 12′, respectively. The probes have conductors 24 and 24′, respectively. In the shown embodiment, there are provided two of the probe holding members, but in other embodiment, a single probe holding member may be provided in the probe holding device according to the present invention.

The probe holding device 10 having the blood flowmeter probes 12 and 12′ is schematically shown in FIG. 2 when viewing along a direction of the arrow A. In the shown embodiment, the probes 12 and 12′ are placed in concave portions of the probe holding members 14 and 14′ so that surfaces of the probe holding members are substantially flush with surfaces of the probes. In the shown embodiment, the leg portions of the “U” shape spread out toward their ends, but they may extend substantially parallel. Alternatively, they may extend while narrowing toward their ends. It is noted that the embodiment shown in FIGS. 1 and 2, the material which forms the probe holding members is translucent or opaque, and therefore the member(s) which is not directly visible is shown with a broken line(s).

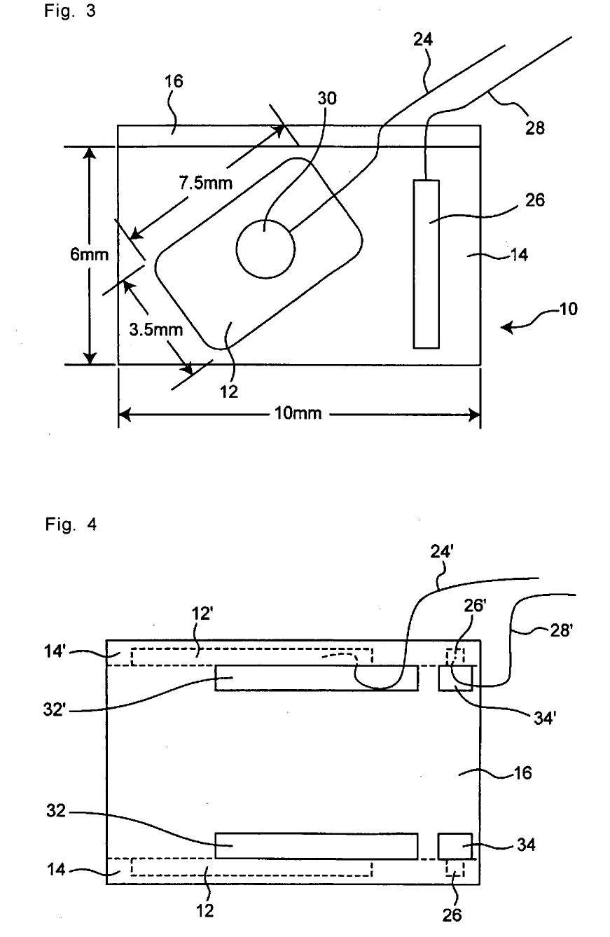

The probe holding device which includes the blood flowmeter probes is schematically shown in FIG. 3 when viewing along a direction shown with the arrow B in FIG. 1. It is noted that the device is shown which further comprises a temperature sensor(s) 26. When viewing along the direction of the arrow B, the temperature sensor 26 and its conductor 28 as well as the blood flowmeter probe 12 and its conductor 24 are not actually visible since they are located on the back side of the probe holding member 14, but they are indicated with solid lines. The probe 12 also comprises a light or sound irradiating and receiving portion 30. One example of sizes of the probe holding member 14 and the probe 12 for the application to a rat is indicated in FIG. 3. It is noted that a thickness of the probe holding members 14 and 14′ is for example 2.0 mm, and a thickness of the probes 12 and 12′ is for example 1.0 mm.

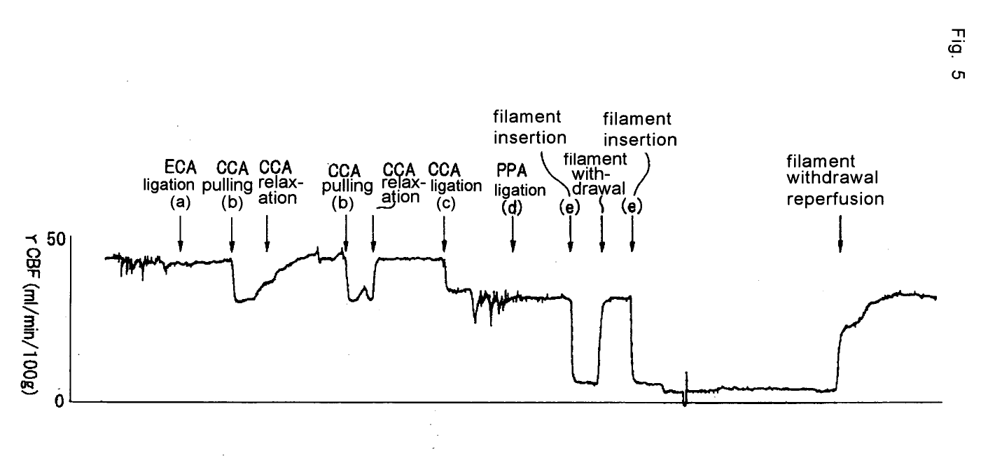

The probe holding device shown in FIG. 1 is schematically shown in FIG. 4 when viewing from the above of the device shown in FIG. 1. It is noted that the bridging part 16 has openings 32, 32′, 34 and 34′ are provided in the embodiment shown in FIG. 4. When the probes 12 and 12′ are located in the concave portions of the probe holding members 14 and 14′, they can reach those portions through the openings 32 and 32′ through the bridging part 16. Similarly, the temperature sensors 26 and 26′ can be provided to the probe holding members through the openings 34 and 34′. It is noted that the conductors 24 and 26 are omitted in FIG. 4.

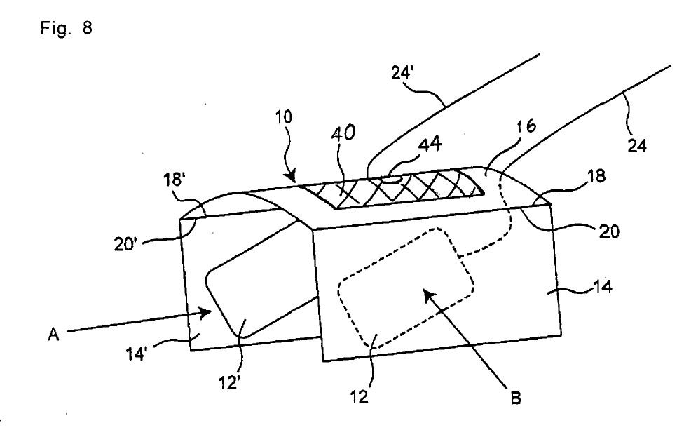

In other embodiment according to the present invention, the bridging part of the probe holding device comprises a heating element which heats a brain. Such embodiment is shown in FIG. 8. In the shown embodiment, the bridging part 16 which connects the probe holding members in the device shown in FIG. 1 comprises the heating element (not shown) in the area of the shaded portion 40. It is sufficient for the heating element that it can electrically heat a portion of the shaded portion area, and optionally it can heat a broader area which may be a whole of such area. More concretely, the heating element is an electrode or a resistor in the form of a plane or a wire, and it is thus preferable that the bridging part is made of an electrically insulating material, for example a plastic material. In the case of the wire form heating element, the heating element may extend in a zig-zag manner or a spiral manner in the area 40. It is noted that current required for heating is supplied through a conductor (not shown).

In one preferable embodiment, the heating element is located on an outer surface of the bridging part, and it is coated with a resin (for example, a curable resin) so as to be electrically insulated. In addition, it is preferable that a temperature sensor 44 (of which conductor is not shown) is provided in the bridging part so as to measure a brain temperature and control thermal dose with the heating element (for example by adjusting the current to be supplied to the heating element) depending on the measured temperature so that the brain temperature can be kept as predetermined. In other embodiment, the temperature sensors 26 and 26′ are used in place of the temperature sensor 44. In a further embodiment, the temperature sensor 44 is provided in addition to the temperature sensor(s) 26 and/or 26′. The manner in which the thermal dose is controlled depending on the temperature as described above is well known, and the means for such controlling is also well known.

When the heating element and the temperature sensor(s) are provided, the brain temperature is directly measured upon the blood flow measurement, so that keeping the brain temperature as predetermined becomes easy.

In order that a brain temperature of a small animal is kept as predetermined under anesthesia, a heating manner has been conventionally employed in which the animal is placed under an infrared lamp on a blanket having an internal heater so as to warm the brain. This manner, however, warms the brain indirectly by placing a whole of the animal under a heat source, and temperature control of the brain in this manner is not easy and thermal dose to be supplied cannot be increased excessively, so that the brain temperature is often lower than an aimed temperature.

In contrast, by providing the heating element in the bridging part, it is possible to keep and control a temperature locally so that the brain temperature can specifically be controlled. Further, there is the following as a very characteristic matter: it has been possible for the conventional heating manner only to control a temperature of an animal which is immobile under anesthesia, and the provision of the heating element to the probe holding device according to the present invention allows the bridging part to be located on the skull while the probe holding members are substantially fixed to sides of the temporal bones, so that in addition to the blood flow, the brain temperature can be continuously monitored and controlled by adjusting the thermal dose to be supplied even when applied to an awaking animal (namely, even when applied to a moving around animal) within its rearing cage. Therefore, the probe holding device according to the present invention can satisfy conflicting conditions required in experiments in which high accuracy is intended while keeping degree of animal freedom high.

It is noted that when the blood flowmeter probe does not have to be provided in the probe holding device when the blood flowmetry is not required but only the brain temperature control is required, only the heating element and the temperature sensor may be provided to the bridging part. In this case, the probe holding device may be referred to as a brain temperature controlling device.

### EXAMPLES

### Example 1

The blood flowmeter probe was provided to the probe holding device according to the present invention as described above so as to form the blood flowmetry device, with which the MCAO model experiments were carried out using rats as follows. It is noted that the device used was as shown in FIGS. 1 and 2, but it comprised only one prove holding member. That is, the device was composed of only the blood flowmeter probe 12 and the probe holding member 14. A conductor 24 was connected to the blood flowmeter.

1. Preparation for Surgery

The rats were anesthetized with inhaled 5% concentration of isoflurane in oxygen. The trachea was then intubated and lungs were mechanically ventilated with a carrier gas of 30% oxygen and 70% nitrogen. The end-tidal concentration of isoflurane was reduced to 2.5%. The pericranial temperature was automatically controlled to 37.0° C. (Mon-a-therm 7000 of Mallinckrodt Inc. was used) by surface heating or cooling. A cannula was inserted in the tail artery with a polyethylene catheter. Arterial pressure was monitored throughout the following MCAO procedure and arterial blood was intermittently sampled to check blood gas, blood glucose, and hematocrit.

2. MCAO Preparation

All rats were surgically prepared for MCAO according to the technique of Zea-Longa. Under an operating scope, a common carotid artery (CCA) was exposed via a midline pretracheal incision. The vagus and sympathetic nerves were separated carefully from the artery. The external carotid artery (ECA) was ligated 2 mm distal to the bifurcation of the common carotid artery. The internal carotid artery was dissected distally to expose the origin of the pterygopalatine artery (PPA).

The common carotid artery (CCA) was then ligated permanently 5-10 mm proximal to its bifurcation and the pterygopalatine artery was ligated close to its origin with a 5-0 nylon monofilament suture. Baseline values for arterial oxygen (PaO₂) and carbon dioxide (PaCO₂) tensions and pH, plasma glucose concentration, hematocrit, systolic arterial pressure, and heart rate were determined. A 0.25 mm-diameter nylon monofilament coated with silicone was introduced into the proximal site of the right common carotid artery via a small arteriotomy.

In the first group of the rats (12 rats), the MCAO was carried out by an examiner with an only 4 weeks experience of making MCAO model and with no LDF monitoring. As described in non-patent references 1 and 2 above mentioned, the filament was advanced about 18-22 mm from the carotid artery bifurcation into the internal carotid artery until there was slight resistance, while in the second group of the rats (12 rats), the same examiner carried out the MCAO with LDF monitoring as described below.

3. rCBF Monitoring by LDF in the Second Group

In Group 2, the blood flowmetry device according to the present invention in the form of a flat rectangular sheet in which a thin probe of the LDF (ADF-21, Advance Co, Inc, Tokyo, Japan) was provided was positioned between the temporal muscle and the lateral aspect of the skull before MCAO preparation on the cerebral cortex of the right hemisphere in the supply territory of the right MCA, so that ultrasonic can be irradiated toward the brain.

The rectangular sheet was made of a polypropylene and had a size of 7.5 mm×3.5 mm×1.0 mm (in thickness). The sheet had concave portions which were complementary to the prove and the conductor so that they were press or snap-fitted into the concave portions.

The used probe was developed for spinal cord blood flow monitoring (available as Type-CS from Unique Medical Inc., Tokyo, Japan). The rectangular sheet was placed in the natural pocket between the temporal muscle and the lateral aspect of the skull after exposing the skull by incision of the skull tissue of the rat, so that the ultrasonic generated by the probe was directed to the brain. Then, after suturing the temporal muscle and connecting tissue on the skull while the temporal muscle was fored to the lateral aspect of the skull through the sheet, the rats were turned upside down to create the MCAO model in the supine position.

rCBF was monitored continuously with 1.0 s. of time constant from before the start for the MCAO operation until 30 min. after the reperfusion. A silicone-coated 4-0 filament was advanced as an intraluminal filament until the laser-Doppler signal decreased by approximately 20% of the base line value. If the laser-Doppler signal showed a steep increase in blood flow during the occlusion period, premature reperfusion was suspected and the position of the filament was readjusted.

In both groups, the end-tidal concentration of isoflurane was reduced to 1.0% during the ischemic period. The filament was withdrawn from the common carotid artery at the end of the 45-min. ischemic period. Thirty minutes after the reperfusion, the tail artery cannula and the LDF probe (only in the second group rats) were removed, the wounds were re-sutured, and then the delivery of isoflurane was stopped. After confirming the resumption of spontaneous ventilation, the mechanical ventilator was disconnected, and the endotracheal tube was removed.

The rats were transferred to a heated and humidified incubator, into which oxygen was delivered constantly. The rats were then allowed to awake from the anesthesia in the incubator and were cared for during the subsequent 2 days before the histological brain examination.

Neurological evaluation was performed two days after the induction of ischemia. The rats were anesthetized with an inhaled 5% concentration isoflurane in oxygen and decapitated. The brains were quickly removed and inspected for the absence of subarachnoid hemorrhage. The brains were sectioned coronally with a tissue chopper at 1-mm intervals, and incubated for 20 min. in a 2% solution of TTC (triphenyl tetrazolium chloride) for vital staining.

The brain sections stained with TTC were recorded with a 3-CCD color video camera (PDMC Ie, Polaroid Co, Inc) to measure the lesion areas. Areas not stained red with TTC, which were considered lesions, were calculated by the video image analyzing system (NIH Image, version 1.52). The total lesion volume (in mm³) was calculated using numerical integration of the TTC-stained areas for all of the sections per rat and the thickness of the sections.

An unpaired t test was used to assess the significance of differences in the physiological variables and lesion volumes between the groups. A paired t test was performed to assess the LDF change. All values presented in the graph (FIG. 7) are the mean +/− SD (standard deviation). A two-tailed value of p <0.05 was considered to be significant.

4. Results

All the physiological variables remained within the normal limits. There were no statistically significant differences in the blood pressure, the arterial blood gases, or the plasma glucose concentration between the two groups throughout the experiments. Three rats in the first group died within 48 hrs. after the MCA occlusion (mortality rate, 3/12=25%), and therefore those rats were excluded from the histopathological analysis. All rats in the second group survived for 48 hrs. after the MCA occlusion. No subarachnoid hemorrhage was observed in the surviving rats while it was present in two of the three dead rats in the first group.

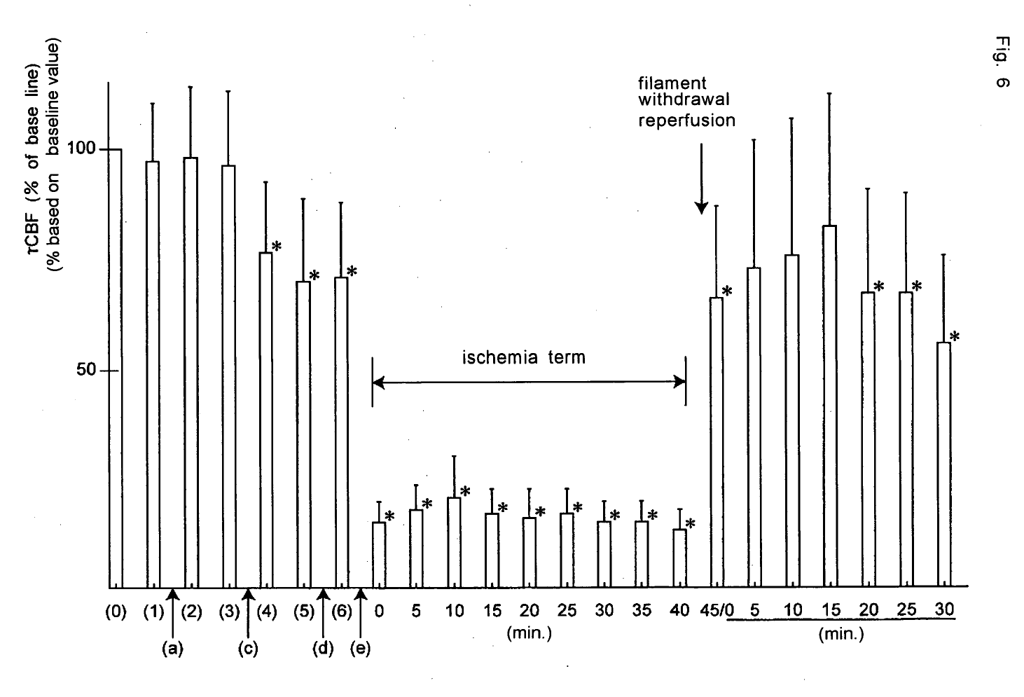

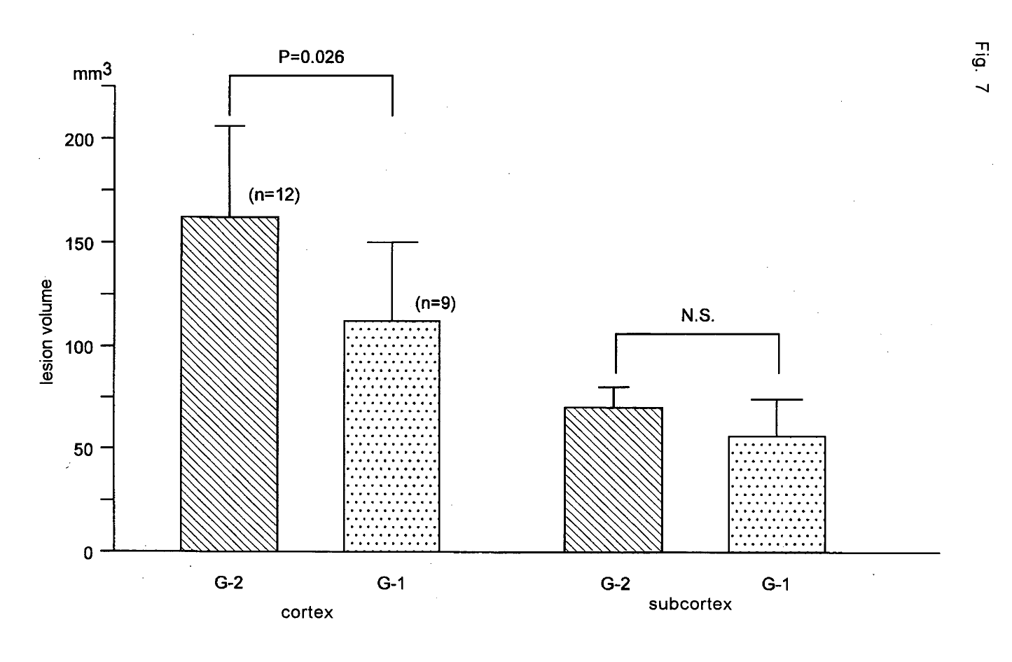

A representative real recording of rCBF detected by the LDF is shown in FIG. 5, in which the ordinate axis indicates rCBF (per unit brain weight and also per unit time) and the abscissa axis indicates the time similarly to FIG. 6 which will be described later. The rCBF was decreased by both pulling ((b) in FIG. 5) and also ligation ((c) in FIG. 5) of the CCA and further an advanced filament ((e) in FIG. 5), while ligation of ECA ((a) in FIG. 5) and PPA ((d) in FIG. 5), which deliver extracranial blood flow, showed no dip in the LDF value, respectively. FIG. 5 shows that using the probe holding device according to the present invention allows the rCBF to be monitored which changes correspondingly and also not correspondingly to pulling, the relaxation, the ligation, and the insertion and the withdrawal of the filament. This means that the rCBF can appropriately be determined by the probe holding device according to the present invention.

FIG. 6 shows the dynamic changes in the rCBF observed by the LDF in the second group. In FIG. 6, the ordinate axis indicates rCBF similarly to FIG. 5 (with the ordinate axis indicating a ratio to the baseline values) and the abscissa axis indicates. In FIG. 6, “*” indicates a significant decrease with respect to the baseline value, and also the width of the standard deviation is shown. The rCBF was decreased both after the CCA ligation (FIG. 3-(4)) by 22+/−12% ((4) in FIG. 6) of the baseline value and after advance of the filament (arrow in FIG. 6) by 80+/−10% of the baseline value, while the ligation of the ECA ((2) in FIG. 6) or the PPA ((6) in FIG. 6) showed no change from their previous values. FIG. 6 shows that the cerebral blood flowmetry achieved by the present invention is highly reproducible.

FIG. 7 shows the calculation results of the lesion volumes of the cortex and the subcortex as described above. The ordinate axis indicates the lesion volume. The lesion volume of the cortex in the second group was 167.21+/−48.54 mm³ (mean +/− standard deviation) significantly, which was considerably larger than that in the first group of 112.77+/−36.03 mm³, P=0.026). The coefficient variation of the lesion volume of the cortex was smaller in the second group (31%) than in the first group (35%), which suggests the better reproducibility of the lesion volume in the second group than in the first group.

The lesion volume of subcortex was similar in both of the first and second groups (the first group: 71.90+/−9.68 mm³ vs. the second group: 59.68+/−21.77 mm³, P=0.57), however, the coefficient variation of the lesion volume of the subcortex was smaller in the second group (13%) than in the first group (36%).

### Example 2

As shown in FIG. 8, a device was produced by positioning, in a zigzag manner, an electrical resistor having a line form as a heating element as well as a temperature sensor in the area 40 of the bridging part 16 of the probe holding device 10 followed by covering them with a silicone resin. The skull by incision of the skull tissue of the rat was exposed similarly to the above, followed by positioning the probe holding members in the both side natural pockets each between the temporal muscle and the lateral aspect of the skull, so that thus produced device was attached to the rat.

The heating was controlled so as to keep the detected temperature of the temperature sensor at 37.0° C. During the three and a half hours of the oxygen-air-isoflurane anesthesia, a rectal temperature of the rat was lowered to 34.5° C. from the initial temperature of 37° C. while the temperature under the temporal muscle was at lowest 36.8° C. so that it has been confirmed that the brain temperature was kept good.

### INDUSTRIAL APPLICABILITY

The device according to the present invention can be very readily mounted onto an animal such as a rat when the intracerebral flowmetry is carried out, and therefore the Dopper blood flowmeter can be easily used for the MCAO model, so that the reproducibility and also the reliability of the experiment are improved. Therefore, the whole of the experiment can be completed in a short term with a less expensive cost.

### CROSS-REFERENCE RELATED TO APPLICATION

The present application claims a priority under the Paris Convention based on Japanese Patent Application No. 2003-414819 (filing date: Dec. 12, 2003, title of the invention: Intracerebral Blood Flow Measuring Device), and the contents described in said application are incorporated herein by reference in their entirety.

1. A probe holding device which includes a probe holding member for holding a blood flowmeter probe and which is used with the blood flowmeter probe when intracerebral blood flow is measured, wherein the probe holding member is allowed to be disposed in a position of being adjacent to and outside a temporal bone while the blood flowmeter probe is held by the member.

2. The device according to claim 1 wherein it comprises two probe holding members, and it further comprises a bridging part which bridges said probe holding members together.

3. The device according to claim 2 wherein the probe holding members and the bridging part are in the form of a sheet respectively, and an edge portion of each probe holding member is connected together to either edge portion of the bridging part.

4. The device according to claim 3 wherein it has a U-shape cross section in which the bridging part corresponds to a bottom bar of the U-shape cross section and the probe holding members correspond to legs of the U-shape cross section which extend from both ends of the bottom bar.

5. The device according to claim 4 wherein the U-shape cross section is provided by folding a sheet material.

6. The device according to claim 1 wherein the device is formed of a plastic material.

7. The device according to claim 1 wherein the probe holding member has a concave portion which is complementary to the form of the prove so that the probe can be fitted into the concave portion.

8. The device according to claim 1 wherein the probe holding member is able to hold also a temperature sensor.

9. The device according to claim 1 wherein the blood flowmeter probe is a probe for the laser-Doppler flowmetry.

10. The device according to claim 1 wherein the blood flowmeter probe is a probe for the ultrasonic-Doppler flowmetry.

11. The device according to claim 1 wherein the probe holding member has a size which allows the member to be positioned between a temporal muscle and a temporal bone.

12. The device according to claim 1 wherein the probe holding member has a size which allows the member to be positioned between a temporal muscle and a temporal bone of a rat or a mouse.

13. The device according to claim 2 wherein the bridging part further comprises a heating element.

14. A blood flow measuring device which comprises
(1) the probe holding device according to claim 1, and
(2) the blood flowmeter probe.

15. The blood flow measuring device according to claim 14 wherein the blood flowmeter probe is a probe for the laser-Doppler flowmetry or the ultrasonic-Doppler flowmetry.

16. The blood flow measuring device according to claim 15 wherein the probe holding device further comprises a temperature sensor.

17. A production process for the probe holding member which is used for the probe holding device according to claim 1, comprising
obtaining a master model which corresponds to a space defined by and between a temporal bone and a temporal muscle, and
then, molding a plastic material based on the obtained master model.

18. The production process according to claim 17 wherein the master model is obtained by pouring a curable material into the space defined by the temporal bone and the temporal muscle followed by curing the curable material in the space.

19. The production process according to claim 18 wherein the curable material is a silicone resin.
